(19) United States
(12) Patent Application Publication
(10) Pub. No.: US 2022/0157423 A1
(43) Pub. Date: May 19, 2022

(54) INTRAOPERATIVE CLINICAL DECISION SUPPORT SYSTEM

(71) Applicants: THE GENERAL HOSPITAL CORPORATION, Boston, MA (US); THE BRIGHAM AND WOMEN'S HOSPITAL, INC., Boston, MA (US)

(72) Inventors: Karen C. Nanji, Brookline, MA (US); David J. Bates, Watertown, MA (US)

(21) Appl. No.: 17/598,387

(22) PCT Filed: Mar. 27, 2020
(86) PCT No.: PCT/US2020/025114

§ 371 Date: Sep. 27, 2021

Related U.S. Application Data
(60) Provisional application No. 62/824,499, filed on Mar. 27, 2019.

(51) Int. Cl. G16H 10/60; G16H 20/17; G16H 50/20; G16H 50/70; A61B 5/0205; A61B 5/00; A61M 16/00; A61M 16/01

(52) U.S. Cl. CPC G16H 10/60; G16H 20/17; G16H 50/20; G16H 50/70; A61B 5/0205; A61B 5/4821; A61M 2016/102; A61M 16/0051; A61M 16/01; A61M 16/024; A61M 2205/3561; A61M 2016/0027; A61B 5/7267

(57) ABSTRACT

Systems and methods are provided for intraoperative real-time clinical decision support. A system includes a processor, a non-transitory computer readable medium, storing executable instructions, and an output device. An interface is configured to receive a first set of patient data in real-time from at least one sensor monitoring vital signals of a patient during a surgical procedure and a second set of patient data in real-time from an anesthesia machine during the surgical procedure. An input interface receives an indicator representing at least an identity of a therapeutic to be administered to the patient. A machine learning model determines from the indicator, the first set of patient data, and the second set of patient data if an alert should be provided to a user. The output device provides the alert to the user if the machine learning model determines that the alert should be provided.

### CROSS-REFERENCE TO RELATED APPLICATION

This application claims the benefit of U.S. Provisional Patent Application No. 62/824,499 filed on Mar. 27, 2019, and entitled PERIOPERATIVE CLINICAL DECISION SUPPORT, which is incorporated herein by reference in its entirety.

### TECHNICAL FIELD

This disclosure relates to clinical decision support systems and is specifically directed to a real-time intraoperative clinical decision support system.

### BACKGROUND

Growing evidence indicates that medication errors and adverse drug events are as common in the perioperative setting as they are throughout the rest of the hospital. While perioperative medication error rates are consistent with rates in other hospital areas, such as inpatient wards and outpatient clinics, the number of medications given in the operating room is so high that every second operation may contain a medication error or an adverse drug event. Almost half of these involve observed patient harm, and the remainder have the potential for harm. Thus, preventing medication errors in the operating room is of great public health importance and has increasingly become a priority locally, regionally, federally, and internationally.

Medication use in the perioperative setting presents particular patient safety challenges. Unlike in the inpatient ward setting, perioperative medication use today usually bypasses standard safety checks, such as electronic physician order entry with decision support, pharmacy approval of drugs, and redundant nursing checks prior to medication administration. In fact, the operating room is one of the few areas in the hospital where every step of the medication use process (i.e., drug selection, dispensing, preparation, administration, documentation, and monitoring) is completed by a single provider, the clinician providing anesthesia, without safety checks by a second person or clinical decision support with alerts to warn of medication errors.

Three main features of the operating room environment limit the use of traditional medication-related electronic clinical decision support today. First, there are typically no prospective medication orders in the operating room. Instead of a physician placing medication orders prior to medication administration, anesthesiologists in the operating room typically select, obtain, prepare, and administer a medication prior to documenting that medication in a one-step order/documentation process that occurs after a medication is administered. Recording medication in the anesthesia information management system (AIMS) functions as both a retrospective order and documentation that the medication was given. Second, patients undergoing surgery are often among the highest acuity patients at the hospital, and they frequently have multiple comorbidities, including cardiovascular and pulmonary disease. Third, due to the nature of anesthesia and the potency of medications administered in the operating room, patients' conditions can quickly change from stable to unstable or vice versa, while under anesthesia.

While there is widespread agreement that user-centered design should be used in electronic health record (EHR) systems, and if it is incorporated, the EHR system is more likely to be effective, user-centered design processes are often not followed. EHR systems are now widely used in the U.S., but there have been broad concerns about their usability and whether this may be causing burnout among providers. One place that EHR systems have been used less often for many tasks and functions, such as clinical decision support and alerts, is in perioperative settings, in part because of the pace of care and the nontraditional medication process in operating rooms. Standardization through electronic clinical decision support has been shown to reduce medication errors in many clinical locations outside of the operating room, including inpatient wards, outpatient clinics, emergency rooms, intensive care units, and long term care facilities. However, intraoperative clinical decision support is currently limited, even where present, and does not provide feedback at a rate useful for the operating room environment.

### SUMMARY

In one example, a system includes a processor, a non-transitory computer readable medium, storing executable instructions, and an output device. The executable instructions include an interface configured to receive a first set of patient data in real-time from at least one sensor monitoring vital signals of a patient during a surgical procedure and a second set of patient data in real-time from an anesthesia machine during the surgical procedure. An input interface receives an indicator representing at least an identity of a therapeutic to be administered to the patient. A machine learning model determines from the indicator, the first set of patient data, and the second set of patient data if an alert should be provided to a user. The output device provides the alert to the user if the machine learning model determines that the alert should be provided to the user.

In another example, a method includes receiving patient data from one of a sensor monitoring a patient during a surgical procedure, an anesthesia machine, and an electronic health records database and an indicator representing at least an identity of a therapeutic to be administered to the patient. A rule set is selected from a plurality of rule sets according to the identity of the therapeutic to be administered to the patient. The rule set is evaluated according to the patient data to determine if a user should be alerted, and the user is alerted if the rule set so indicates. The identity of the therapeutic is then sent to an associated system for documentation.

In a further example, a system includes a processor, a non-transitory computer readable medium, storing executable instructions, and an output device. The executable instructions include an interface configured to receive a first set of patient data in real-time from at least one sensor monitoring vital signals of a patient during a surgical procedure and a second set of data in real-time from an anesthesia machine. An anesthesia information management system (AIMS) interface is configured to receive a third set of patient data from an AIMS during the surgical procedure. A network interface is configured to retrieve a fourth set of patient data associated with the patient from an electronic health records database. An input interface receives an indicator representing at least an identity of a therapeutic to be administered to the patient. A rule-based expert system includes a plurality of rule sets. Each rule set determines if an alert should be provided to a user from at least one of the indicator, the first set of patient data, the second set of patient data, and the third set of patient data.

A retraining engine retrains at least a subset of the plurality of rule sets according to a set of labelled training samples. Each training sample includes at least a portion of the data comprising the indicator, the first set of patient data, the second set of patient data, and the third set of patient data, as well as a label derived from at least one of patient outcomes retrieved from the electronic health records database and disagreements indicated by users. The output device provides the alert to the user if a rule set of the plurality of rule sets determines that the alert should be provided to the user. The user can indicate disagreement with the alert via the input interface.

### BRIEF DESCRIPTION OF THE DRAWINGS

FIG. 1 depicts an example of a clinical decision support (CDS) system.

FIG. 2 depicts one implementation of a CDS server that can be used in a CDS system.

FIG. 3 illustrates a sample dosing window with a dosing alert from an example CDS system.

FIG. 4 illustrates an alert window from an example CDS system.

FIG. 5 illustrates one example of a method for providing intraoperative clinical decision support.

FIG. 6 is a schematic block diagram illustrating an exemplary system of hardware components capable of implementing examples of the systems and methods disclosed in FIGS. 1-5.

### DETAILED DESCRIPTION

As used in this application, “patient data” refers to medically relevant data associated with the patient. This can include, but is not limited to, patient characteristics, such as age, sex, height, and weight; laboratory values such as blood glucose level, blood chemistry panels, complete blood counts, blood gas levels, urinalysis, culture results; and clinical measurements such as blood pressure, heart rate, oxygen saturation, exhalatory anesthetic agent volume and content, and respiratory rate, and medical history, including past values for clinical measurements and patient characteristics, diagnoses of medical conditions, allergies, and current and past therapeutic interventions performed on the patient.

A “therapeutic,” is a medication, an other therapeutic substance, such as blood or intravenous fluid, or a therapeutic action, which can include, but is not limited to, taking bloodwork, monitoring the patient, adjusting the sensors monitoring the patient, consulting other professional staff, suctioning the patient, adjusting pressure points, or starting chest compressions in cardiac arrest.

Data is provided in “real-time” only if it has a latency of less than ten seconds.

A “range” can have two bounding values (e.g., between five and ten milligrams) or a single explicit bounding value (e.g., less than ten milligrams).

An “alert” is a visual, auditory, or tactile notification provided to a user via an output device. An alert can, but does not necessarily, include information explaining a rationale for the alert or a prompt to allow the user to retrieve information related to the rationale for the alert.

This disclosure relates to systems and methods for providing intraoperative clinical decision support. It is especially important in the fast-paced, rapidly changing operating room environment that alerts from a clinical decision support (CDS) system be able to incorporate real-time patient data such as vital signs and medications being administered. For example, the CDS system should capture whether medications are incompatible with a patient's vital signs at a particular second in time or whether there may be a drug-drug interaction with a prior medication that was administered thirty seconds ago. Most anesthesia information management systems do not have access to real-time vital sign data—they typically collect these data averaged over one minute, which is too slow for effective medication-related clinical decision support in the operating room.

Delivery of anesthesia is often tailored to patient and provider preferences. Anesthesiologists may titrate doses to effect while at the same time considering a patient's physiologic limitations such as renal insufficiency, pain, or coronary artery disease, making too much standardization of practice challenging and undesirable. Medication-related CDS alerts must be tailored to patient baseline physiology, incorporating, for example, baseline blood pressure, oxygen saturation, or renal function. For example, a blood pressure that is low for one patient may be acceptable for another. An antibiotic dose for a patient with normal renal function may too high for a patient with renal insufficiency. In order for CDS to be useful in the OR, it must consider these variations in patient physiology.

Accordingly, the disclosed systems and methods provide real-time intraoperative clinical decision support to enhance patient care and avoid errors due to the fast pace of the operating room environment. The systems and methods can be implemented, for example, as computer-executable instructions implementing one or more applications for performing functions associated with interfacing with various devices, such as patient monitoring equipment, healthcare record keeping systems, and other information sources, conditioning the retrieved data, providing clinical decision support based on the retrieved data, and providing an interface to receive input from and provide information to one or more health professional using the system.

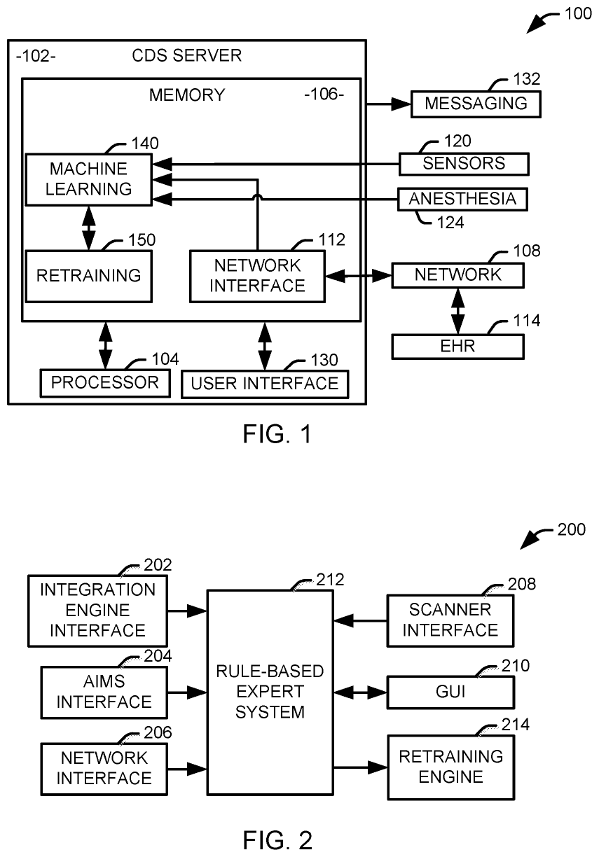

FIG. 1 depicts an example of a clinical decision support (CDS) system 100. In the example of FIG. 1, the CDS system 100 includes a CDS server 102. The server 102 can include one or more processors 104 and a memory 106. It will be appreciated that the memory 106 can comprise one or more discrete units of physical memory operatively connected to process to store data and machine-readable instructions that can be executed by the processor 104. For example, the memory 106 can comprise physical memory, which can reside on the processor 104 (e.g., processor memory), random access memory or other physical storage media (e.g., CD-ROM, DVD, flash drive, hard disc drive, etc.) or a combination of different memory devices that can store the executable instructions. The data utilized for implementing the systems and methods described herein can also be stored in the memory 106 or in some other arrangement of one or more memory structures that are accessible for use by the CDS system 100.

The CDS server 102 can be connected to a network 108 via a network interface 112 to provide for communication between the CDS server 102 and various services, devices, and data stores that contain patient data. The network 108 can be a local area network, a wide area network, or a combination of different various network topologies, which may include physical transmission media (e.g., electrically conductive, optical fiber media or the like) and/or wireless communications media, that can be utilized for communicating information. The network 108, or at least a portion of the methods and functions implemented thereby, can operate in a secure manner (e.g., behind a firewall separated from public networks) and/or utilize encryption for data communications. In one example, the CDS server 102 comprises a network interface 112 to retrieve patient data from an electronic health records (EHR) database 114. In general, the patient data collected from the electronic health records database 114 includes relatively static characteristics of the patient, such as height, weight, baseline renal function, a baseline blood pressure, chronic conditions, current medications, allergies, and other medical history. Accordingly, these parameters can be retrieved once at the start of a procedure. Laboratory results and other new data can be retrieved from the EHR database continuously through the procedure as well to ensure that decisions are made on the most recent data.

In the example of FIG. 1, the CDS server 102 receives a first set of patient data from a set of sensors 120 connected to a patient to monitor vital signs of the patient in real-time. The first set of patient data can include, for example, systolic blood pressure, mean arterial blood pressure, heart rate, temperature, alerts from an electrocardiograph, and oxygen saturation. The CDS server 102 receives a second set of patient data from an anesthesia machine 124 in real-time. The second set of patient data can include, for example, a respiratory rate, end tidal gas concentrations, a fraction of inhaled oxygen, a tidal volume, an airway pressure, and other respiratory parameters. In one implementation, the real-time data received from the set of sensors 120 and the anesthesia 124 can be received each second. In practice, the CDS server 102 can receive the first and second sets of patient data either directly from the set of sensors 120 and the anesthesia machine 124 or intermediated by an integration engine that collects real-time data from the set of sensors and the anesthesia machine.

A user interface 130 includes an output device, such as a display, speaker, or tactile feedback device, as well as an input device, such as a touchscreen, keyboard, or mouse, microphone, or barcode scanner, to allow information to be exchanged between a user and the CDS server 102. For example, the user interface 130 can be a computer, a work station, or a mobile device, such as a smart phone, laptop, or tablet computer, that can run a corresponding application programmed to access the CDS server 102. In one example, the user interface 130 further includes a scanner device (not shown) for scanning a container holding a therapeutic to obtain an indicator representing an identity of the therapeutic. It will be appreciated, however, that the identity of therapeutics administered to the patient can be entered by other means such as manually by a user via an input device, such as a mouse, keyboard, touchscreen, or microphone, associated with the user interface 130. In one example, the input device associated with the user interface 130 can be used to notify the system that the user has elected to not to follow the recommended action (or inaction) associated with the alert, a situation referred to herein as an “override” of the alert. The CDS server 102 can also employ a messaging system 132 to send messages to respective users via the network interface 108. For example, the messaging system 132 can send any of alphanumeric pages, for example, via a hospital paging system, a text message, an automated phone or voice message, and/or an email message to alert a user of issues with the patient.

The patient data retrieved from the EHR system, the first set of patient data, the second set of patient data, and any indicators representing therapeutics provided to the patient are provided to a machine learning model 140 for analysis. The machine learning model 140 can utilize one or more pattern recognition algorithms, implemented, for example, as classification and regression models, each of which analyze the patient data to determine if an alert should be generated for the user via the user interface 130. In one implementation, multiple classification and regression models are used, with each model providing an “alert” or “normal” class or an index which can be thresholded to indicate when an alert is desirable.

The machine learning model 140 can be trained on training data representing the various classes or dependent variables of interest. The training process of the machine learning model 140 will vary with its implementation, but training generally involves a statistical aggregation of training data into a set of parameters for the model. Any of a variety of techniques can be utilized for the models, including support vector machines, regression models, self-organized maps, k-nearest neighbor classification or regression, fuzzy logic systems, data fusion processes, boosting and bagging methods, rule-based systems, or artificial neural networks.

For example, an SVM classifier can utilize a plurality of functions, referred to as hyperplanes, to conceptually divide boundaries in the N-dimensional feature space, where each of the N dimensions represents one associated feature of the feature vector. The boundaries define a range of feature values associated with each class. Accordingly, an output class and an associated confidence value can be determined for a given input feature vector according to its position in feature space relative to the boundaries. An SVM classifier utilizes a user-specified kernel function to organize training data within a defined feature space. In the most basic implementation, the kernel function can be a radial basis function, although the systems and methods described herein can utilize any of a number of linear or non-linear kernel functions.

An ANN classifier comprises a plurality of nodes having a plurality of interconnections. The values from the feature vector are provided to a plurality of input nodes. The input nodes each provide these input values to layers of one or more intermediate nodes. A given intermediate node receives one or more output values from previous nodes. The received values are weighted according to a series of weights established during the training of the classifier. An intermediate node translates its received values into a single output according to a transfer function at the node. For example, the intermediate node can sum the received values and subject the sum to a binary step function. A final layer of nodes provides the confidence values for the output classes of the ANN, with each node having an associated value representing a confidence for one of the associated output classes of the classifier.

A k-nearest neighbor model populates a feature space with labelled training samples, represented as feature vectors in the feature space. In a classifier model, the training samples are labelled with their associated class, and in a regression model, the training samples are labelled with a value for the dependent variable in the regression. When a new feature vector is provided, a distance metric between the new feature vector and at least a subset of the feature vectors representing the labelled training samples is generated. The labelled training samples are then ranked according to the distance of their feature vectors from the new feature vector, and a number, k, of training samples having the smallest distance from the new feature vector are selected as the nearest neighbors to the new feature vector.

In one example of a classifier model, the class represented by the most labelled training samples in the k nearest neighbors is selected as the class for the new feature vector. In another example, each of the nearest neighbors can be represented by a weight assigned according to their distance from the new feature vector, with the class having the largest aggregate weight assigned to the new feature vector. In a regression model, the dependent variable for the new feature vector can be assigned as the average (e.g., arithmetic mean) of the dependent variables for the k nearest neighbors. As with the classification, this average can be a weighted average using weights assigned according to the distance of the nearest neighbors from the new feature vector. It will be appreciated that k is a metaparameter of the model that is selected according to the specific implementation. The distance metric used to select the nearest neighbors can include a Euclidean distance, a Manhattan distance, or a Mahalanobis distance.

A regression model applies a set of weights to various functions of the extracted features, most commonly linear functions, to provide a continuous result. In general, regression features can be categorical, represented, for example, as zero or one, or continuous. In a logistic regression, the output of the model represents the log odds that the source of the extracted features is a member of a given class. In a binary classification task, these log odds can be used directly as a confidence value for class membership or converted via the logistic function to a probability of class membership given the extracted features.

A rule-based classifier applies a set of logical rules to the extracted features to select an output class. Generally, the rules are applied in order, with the logical result at each step influencing the analysis at later steps. The specific rules and their sequence can be determined from any or all of training data, analogical reasoning from previous cases, or existing domain knowledge. One example of a rule-based classifier is a decision tree algorithm, in which the values of features in a feature set are compared to corresponding threshold in a hierarchical tree structure to select a class for the feature vector. A random forest classifier is a modification of the decision tree algorithm using a bootstrap aggregating, or “bagging” approach. In this approach, multiple decision trees are trained on random samples of the training set, and an average (e.g., mean, median, or mode) result across the plurality of decision trees is returned. For a classification task, the result from each tree would be categorical, and thus a modal outcome can be used, but a continuous parameter can be computed according to a number of decision trees that select a given task. It will be appreciated that the number of trees, as well as a number of features used to generate trees, can be selected as metaparameters for the random forest model.

A retraining engine 150 accumulates feedback from the system to retrain the machine learning model 140. In one example, the retraining engine 150 logs each instance in which a user overrides an alert. The override, as well as the inputs to the machine learning model associated with the alert, can be saved at the retraining engine 150. In one example, all patient data received or generated during a given procedure can be saved whenever an override of an alert is recorded to allow additional features to be added to various classifier systems in the machine learning model. Additionally, or alternatively, the retraining engine 150 can store patient data from all procedures and compare the stored data to patient outcomes recorded in the EHR. Accordingly, the machine learning model 140 can be continuously refined to provide meaningful alerts for a user.

FIG. 2 depicts one implementation of a CDS server 200 that can be used in a CDS system. The CDS server 200 includes an integration engine interface 202 that obtains, in real-time, a first set of patient data, representing real-time vital signs, such as blood pressure, heart rate, and oxygen saturation, and a second set of data, representing a respiratory rate, end tidal gas concentrations, a fraction of inhaled oxygen, a tidal volume, an airway pressure, and other respiratory parameters from an integration engine connected to patient monitors and the anesthesia machine. While this information is available from the anesthesia information management system (AIMS), most AIMS only collect vital signs averaged over a one-minute time period, which could result in alerts falsely firing based on a value from one minute ago that has since been corrected. However, a third set of patient data can be collected from the AIMS an AIMS interface 204. Data collected from the AIMS can include continuously monitored physiologic parameters such as measures of neuromuscular blockade, urine output, and blood loss. Each of the integration engine interface 202 and the AIMS interface 204 can be implemented as an application programming interface (API) configured to receive data from the integration engine and the AIMS, respectively, in a format suitable for the CDS server 200.

Additional patient data can be extracted from the electronic health record (EHR) database via a network interface 206. In one example, at the start of each procedure, patient demographic data (e.g., age, sex, etc.), a problem list, representing existing conditions that are relevant to intraoperative care, allergies, medications, and laboratory values (e.g., creatinine, blood glucose, etc.) are extracted from the EHR. During the procedure, the EHR can be queried periodically for updated data, such as intra-operative laboratory values.

Historically, anesthesia providers have documented medications in the anesthesia information management system only after administration. In the illustrated system 200, initiation of the medication documentation process occurs immediately prior to medication administration instead of the usual post-administration documentation. While this workflow change can be achieved with manual documentation, it is facilitated in the illustrated system via the introduction of point-of-care bar code scanning of syringe labels immediately prior to administering each medication. Bar code scanning and other methods can simplify medication documentation for the user by avoiding having to manually find the correct medication screen in the patient cart.

Accordingly, a scanner interface 208 can receive a scanned value from a bar code captured at a bar code scanner. This value can be provided to a graphical user interface 210, such that the bar code scan immediately prior to administration launches a medication dosing screen populated with the correct medication name and concentration, allowing the provider to just enter the desired dose. The final medication data are then sent to the patient's record in the AIMS via the AIMS interface 204 for documentation in real-time, eliminating the need to manually enter the medication name and dose into the AIMS.

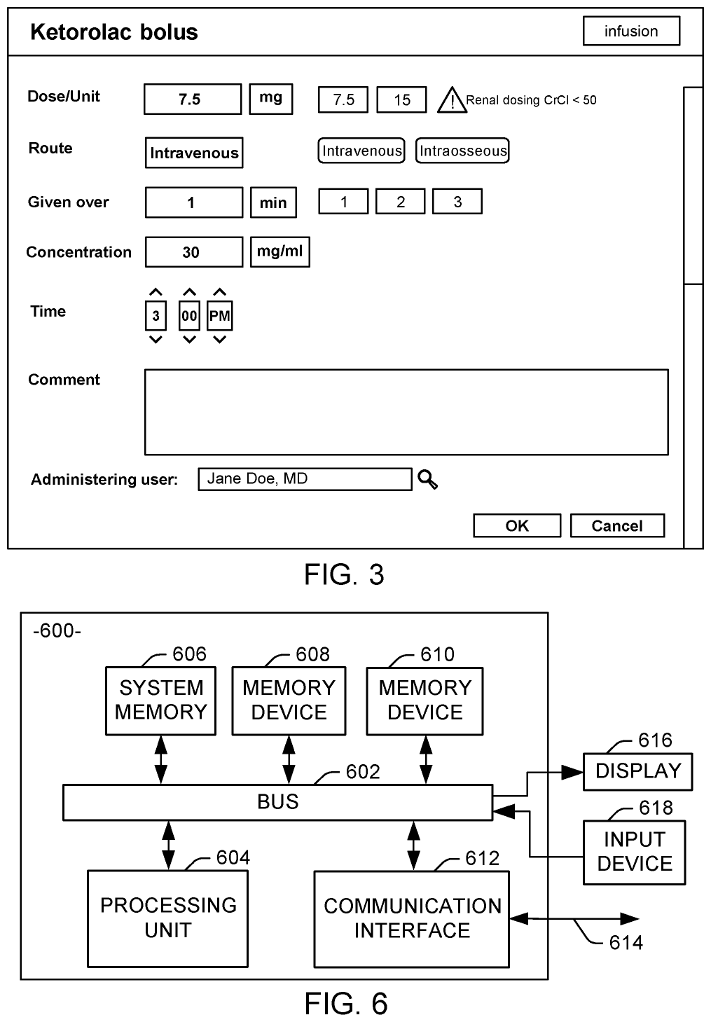

The graphical user interface 210 presents alerts to the user and allows the user to enter medication dosing information via medication dosing windows and alert windows. Medication dosing windows display the medication name and concentration provided by the medication scan and have fields for data input by the user including dose, dosing units, administration time. Default values are included for all fields, and quick-choice buttons are included for the dose field. The dosing windows incorporate non-interruptive alerts, including default doses that are based on weight and/or creatinine clearance where appropriate. A sample dosing window with a non-interruptive alert is illustrated in FIG. 3.

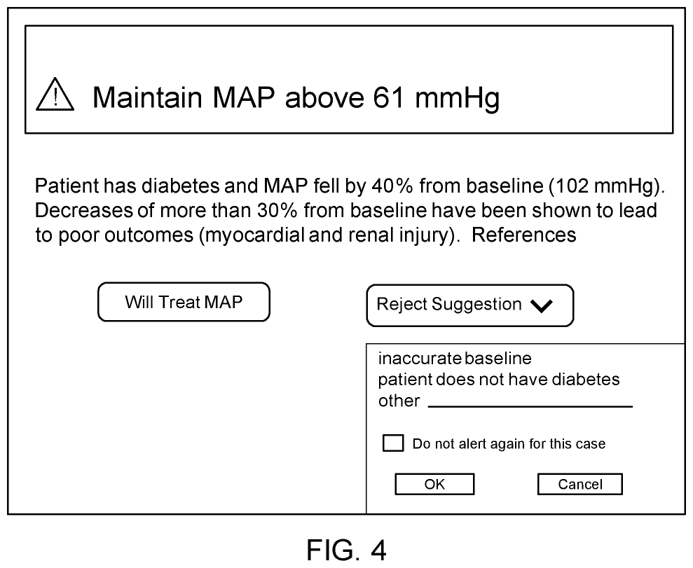

Based on the first set of patient data, the second set of patient data, the data extracted from the EHR, the data from the AIMS interface, and any data received from the bar code scanner and the graphical user interface 210, the CDS server 200 generates interruptive alert windows, where applicable, for critical alerts including errors such as wrong medication, wrong dose, wrong timing and reminder alerts. In the illustrated implementation, each alert window includes an alert title, alert description, rationale, and reference. An example of an alert window is illustrated as FIG. 4. To generate the alerts, a rule-based expert system 212 applies sets of logical rules to the collected patient data, and determines, from the applied sets of logical rules, if a user should be alerted to a situation in the operating room. The alerts associated with the sets of logical rules can be generally categorized as either reminder alerts or medication-triggered alerts.

A reminder alert represents a condition in which intervention by the user is determined to be advisable to avoid harm or risk of harm to the patient. Rule sets related to reminder alerts generally evaluate the data from the monitor and anesthesia machine via the integration engine interface 202 and the AIMS interface 204 to determine if intervention is advisable. In some cases, data retrieved from the EHR system is also used in evaluating the rule set. One general category of rule sets associated with reminder alerts involve comparison of a vital sign or a value from the AIMS with a range of values and alerting the user if the vital signal or AIMS value falls in the range of values. This value can be individualized for the user according to other characteristics of the patient, include demographic parameters, preexisting conditions, other therapeutics administered, or any of a number of other parameters. It will be appreciated that this category of alerts can be checked continuously throughout a surgical procedure and does not require a specific trigger.

In one example, reminder alerts are used for low oxygen saturation, hypotension, hypertension, and low heart rate. The ranges can be predefined or defined according to preexisting conditions of the patient. Another set of rules provide additional alerts when a value has fallen into a prescribed range for more than a threshold period of time. These rule sets can be evaluated only when a value falls within the defined range. A third set of reminder alerts notify a user of missing data or actions in the patient record. These can be conditioned on a preexisting medical condition (e.g., alert if a patient on a specific medication does not have a result for a particular test in the record) or applied to all patients (e.g., alert if no blood pressure taken prior to induction or if no blood pressure value has been received within a predetermined period of time). Depending on the specific rule, these rule sets can be evaluated once at the beginning of a procedure, at periodic intervals, or continuously throughout the procedure. Finally, abnormal conditions from various sensors (e.g., arrhythmias from ECGs) can be tracked at the rule-based expert system 212 and an alert can be provided to the user when a specified condition is met.

A second set of alerts are triggered by administration of a therapeutic. It will be appreciated that the rule set for each therapeutic-triggered alert can be associated with a specific therapeutic or class of therapeutics, such that it is only necessary to evaluate the rule set when an associated therapeutic is to be administered. A first variety of these therapeutic-triggered alerts schedules a follow-up action after a therapeutic is administered and notifies a user if the follow-up action is not taken within a specified period of time. This can be a general alert for all medications, (e.g., a reminder to document the administration of the medication), for a category of medications, (e.g., a reminder to resecure containers of controlled substances after administration), or for a specific medication (e.g., a reminder to check glucose levels after administering insulin). These alerts can also be conditional based upon other patient data, such that the alert is only provided for patients having an underlying condition, a previous therapeutic intervention, or a measured or calculated metric within a predetermined range.

A second variety of therapeutic-triggered alerts provide reminders to the user to modify the dose given to the patient based on an existing medical condition of the patient, a previous therapeutic intervention, or a measured or calculated metric within a predetermined range. These reminders can be given, for example, at a screen used to enter the dosage to be given to the patient. Finally, a third variety of therapeutic-triggered alerts determine if it is appropriate to give a medication to the patient. Each rule set can evaluate the dosage entered by the user, the first and second sets of patient data, any local data stored at the CDS server 200 (e.g., representing previous alerts and previous therapeutics or other interventions provided to the patient), and any patient data retrieved from the EHR. An alert can be provided to the user before administration of the therapeutic if a rule set determines that it is inadvisable to provide the therapeutic in the dosage entered.

Upon receiving an alert, the provider may decide to accept the alert and revise the action that generated the alert or override the alert and continue with the planned action. Alert overrides can require the user to indicate a reason for override. In one implementation, alerts that have high levels of appropriate overrides can be modified or deleted, alerts with high levels of inappropriate overrides are good targets for provider educational interventions, and alerts with high levels of acceptance can remain.

To this end, a retraining engine 214 accumulates feedback from the system to refine the sets of rules at the rule-based expert system 212. In one example, the retraining engine 214 logs each instance in which a user overrides an alert. The override, as well as any relevant parameters associated with the alert (or associated with relevant patient outcomes), can be saved at the learning model. It will be appreciated that the relevant parameters can include not only parameters evaluated by the rule, but a set of additional parameters indicated in the learning model to be relevant for a given rule set. For example, if the rule set determines if a heart rate of the patient has fallen below a threshold value, the additional relevant parameters could include past and future measurements of the patient's heart rate, current, past and future measurements of the patient's blood pressure, and any arrhythmias detected on an ECG. In one example, all patient data received or generated during a given procedure can be saved whenever an override of an alert is recorded. Additionally, or alternatively, the retraining engine 214 can store patient data from all procedures and compare the stored data to patient outcomes recorded in the EHR.

It will be appreciated that many of the rule sets include ranges of times, dosages, and measured or calculated parameters, with threshold values bounding one or both sides of the range. Further, while the default rules are the product of clinical judgement, the retraining engine 214 may find unknown interactions that can be represented by the addition of rules to a given rule set. Accordingly, in one example, a decision tree representing the rule set, in which one or both of the patient outcome, expressed as a categorical variable, or the presence or absence of an override for a given alert are used to label training samples associated with a given alert.

During retraining, a set of features relevant to the alert can then be selected. In one example, a univariate approach, such as a Pearson's chi-squared test, can be used to identify features useful for distinguishing between the labelled categories of features. It will be appreciated, however, that multivariate approaches can be used for feature selection, such as a lasso regression or training a random forest classifier on the selected data to determine the relative importance of the features under consideration. Once a set of features have been selected, a decision tree can be trained using the selected features to provide a revised set of rules represented by the decision tree. For example, a globally-optimal classification tree analysis can be used to train the decision tree. In practice, the training of the decision tree can be subject to constraints to preserve the clinical knowledge represented by the existing rule. For example, the decision tree can be constrained to include the one or more parameters currently utilized by the rule, and changes to the threshold values associated with these parameters can be limited to a predefined percentage of the original value.

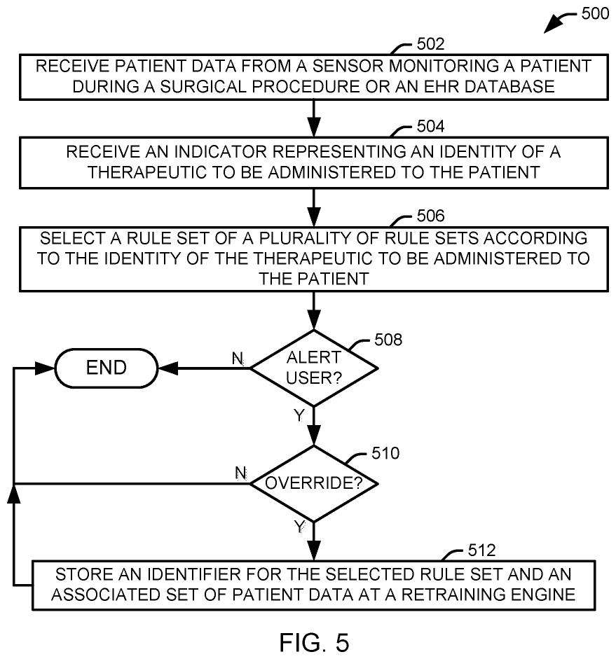

In view of the foregoing structural and functional features described above in FIGS. 1-4, example methods will be better appreciated with reference to FIG. 5. While, for purposes of simplicity of explanation, the method of FIG. 5 is shown and described as executing serially, it is to be understood and appreciated that the present invention is not limited by the illustrated order, as some actions could in other examples occur in different orders and/or concurrently from that shown and described herein.

FIG. 5 illustrates one example of a method 500 for providing intraoperative clinical decision support. At 502, patient data is received from one of a sensor monitoring a patient during a surgical procedure and an electronic health records database. It will be appreciated that the sensor monitoring the patient include sensors for monitoring vital signs of patients, such as sphygmomanometers, pulse oximeters, and ECG systems, as well as sensors associated with an anesthesia machine. In one example, a clinical decision support system that receives the patient data is directly connected to the sensors for monitoring vital signs of patients, such that the vital sign data is provided in real-time. In another implementation, the patient data is retrieved from an integration engine that receives real-time data from the sensors. In some implementations, the sensors provide values for the patient data each second.

At 504, an indicator is received representing at least an identity of a therapeutic to be administered to the patient. In one implementation, the indicator includes both an identity and dosage of the therapeutic, for example, with the identity of the therapeutic provided by scanning a bar code on a container of the therapeutic and a dosage entered by the user via a user interface. The container can be the container in which the therapeutic was purchased, stored, or administered to the patient. At 506, a rule set of a plurality of rule sets is selected according to the identity of the therapeutic to be administered to the patient. At 508, the rule set is evaluated according to the patient data to determine if a user should be alerted. In one implementation, evaluation of the rule set is triggered after the therapeutic is administered to the patient. In this case, the patient data can include a parameter that indicates if an action has been taken by a caregiver, and the rule set determining if an alert is necessary if the first parameter indicates that the action has not been taken within a predetermined time period after administering the therapeutic.

In another implementation, evaluating the rule set includes determining if the therapeutic should be administered to the patient. In one example, where the indicator includes a dosage of the therapeutic, it is determined that the therapeutic should be administered to the patient only if the dosage is within a defined range. The defined range can be predetermined or determined according to a parameter from the patient data, such that a first defined range is used if the parameter assumes a first value and a second defined range is used if the parameter assumes a second value.

In another example, the rule set determines that the therapeutic should not be administered to the patient if a dose of the therapeutic was prepared more than a threshold period of time before it is to be administered. In still another example, it is determined that the therapeutic should not be administered to the patient if another therapeutic is currently being administered to the patient on an intravenous line through which the first therapeutic is to be administered. In yet another example, it is determined that the therapeutic should not be administered to the patient if a parameter from the patient data assumes a specific value or falls within a predefined range. For example, the parameter can be a categorical parameter representing a diagnosis of a medical condition for the patient, either at any time or in a predetermined period before the procedure or a categorical parameter representing administration of a dose of a medication within a predetermined time period before administration of the therapeutic.

If the rule set determines that no alert is necessary (N), the method terminates. If it is determined that a user should be alerted (Y), the method advances to 510 where it is determined if the user has overridden the alert. If no override is received (N), the method terminates. If an override is received (Y), the method advances to 512, where an identifier for the selected rule set and a set of patient data associated with the rule set are stored at a retraining engine. The retraining engine can utilize overrides received by users, as well as recorded patient outcomes, to develop an additional training set for refining the rule set used to generate the alert. The method 500 then terminates.

In one implementation, information, including at least the identity and dosage of the therapeutic, is provided to an associated system (AIMS) for documentation when any therapeutic is administered. In the illustrated example, the information is provided to an AIMS and includes the identity and dosage of the therapeutic, as well as identity of the individual administering the therapeutic and the time at which the therapeutic was administered.

FIG. 6 is a schematic block diagram illustrating an exemplary system 600 of hardware components capable of implementing examples of the systems and methods disclosed in FIGS. 1-5. The system 600 can include various systems and subsystems. The system 600 can be a personal computer, a laptop computer, a workstation, a computer system, an appliance, an application-specific integrated circuit (ASIC), a server, a server blade center, a server farm, etc.

The system 600 can includes a system bus 602, a processing unit 604, a system memory 606, memory devices 608 and 610, a communication interface 612 (e.g., a network interface), a communication link 614, a display 616 (e.g., a video screen), and an input device 618 (e.g., a keyboard and/or a mouse). The system bus 602 can be in communication with the processing unit 604 and the system memory 606. The additional memory devices 608 and 610, such as a hard disk drive, server, stand-alone database, or other non-volatile memory, can also be in communication with the system bus 602. The system bus 602 interconnects the processing unit 604, the memory devices 606-610, the communication interface 612, the display 616, and the input device 618. In some examples, the system bus 602 also interconnects an additional port (not shown), such as a universal serial bus (USB) port.

The processing unit 604 can be a computing device and can include an application-specific integrated circuit (ASIC). The processing unit 604 executes a set of instructions to implement the operations of examples disclosed herein. The processing unit can include a processing core.

The additional memory devices 606, 608, and 610 can store data, programs, instructions, database queries in text or compiled form, and any other information that can be needed to operate a computer. The memories 606, 608 and 610 can be implemented as computer-readable media (integrated or removable) such as a memory card, disk drive, compact disk (CD), or server accessible over a network. In certain examples, the memories 606, 608 and 610 can comprise text, images, video, and/or audio, portions of which can be available in formats comprehensible to human beings. Additionally, or alternatively, the system 600 can access an external data source or query source through the communication interface 612, which can communicate with the system bus 602 and the communication link 614.

In operation, the system 600 can be used to implement one or more parts of a clinical decision support system in accordance with the present invention. Computer executable logic for implementing the clinical decision support system resides on one or more of the system memory 606, and the memory devices 608, 610 in accordance with certain examples. The processing unit 604 executes one or more computer executable instructions originating from the system memory 606 and the memory devices 608 and 610. The term “computer readable medium” as used herein refers to any medium that participates in providing instructions to the processing unit 604 for execution, and it will be appreciated that a computer readable medium can include multiple computer readable media each operatively connected to the processing unit.

Specific details are given in the above description to provide a thorough understanding of the embodiments. However, it is understood that the embodiments can be practiced without these specific details. For example, physical components can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine-readable mediums for storing information. The term “machine-readable medium” includes, but is not limited to portable or fixed storage devices, optical storage devices, wireless channels, and/or various other storage mediums capable of storing that contain or carry instruction(s) and/or data.

What have been described above are examples of the invention. It is, of course, not possible to describe every conceivable combination of components or methodologies, but one of ordinary skill in the art will recognize that many further combinations and permutations of the invention are possible. Accordingly, the invention is intended to embrace all such alterations, modifications and variations that fall within the scope of the appended claims and the application. Additionally, where the disclosure or claims recite “a,” “an,” “a first,” or “another” element, or the equivalent thereof, it should be interpreted to include one or more than one such element, neither requiring nor excluding two or more such elements. As used herein, the term “includes” means includes but not limited to, the term “including” means including but not limited to. The term “based on” means based at least in part on.

What is claimed is:

1. A system comprising:
a processor;
a non-transitory computer readable medium, storing executable instructions, the executable instructions comprising:
an interface configured to receive a first set of patient data in real-time from at least one sensor monitoring vital signals of a patient during a surgical procedure and a second set of patient data in real-time from an anesthesia machine during the surgical procedure;
an input interface that receives an indicator representing at least an identity of a therapeutic to be administered to the patient; and
a machine learning model that determines from the indicator, the first set of patient data, and the second set of patient data if an alert should be provided to a user; and
an output device that provides the alert to the user if the machine learning model determines that the alert should be provided to the user.

2. The system of claim 1, the executable instructions further comprising a network interface configured to retrieve information associated with the patient from an electronic health records database.

3. The system of claim 3, the executable instructions further comprising a retraining engine that retrains the machine learning model according to patient outcomes retrieved from the electronic health records database.

4. The system of claim 1, the executable instructions further comprising a retraining engine that retrains the machine learning model according to feedback from the user given in response to the alert.

5. The system of claim 1, wherein the machine learning model comprises a rule-based expert system, comprising a plurality of rule sets, each rule set determining if an alert should be provided to a user from at least one of the indicator, the first set of patient data, and the second set of patient data.

6. The system of claim 5, wherein the rule-based expert system comprises at least one rule set that determines, from at least the indicator, if the therapeutic should be administered to the patient.

7. A method comprising:
receiving patient data from one of a sensor monitoring a patient during a surgical procedure, an anesthesia machine, and an electronic health records database;
receiving an indicator representing at least an identity of a therapeutic to be administered to the patient; and
selecting a rule set of a plurality of rule sets according to the identity of the therapeutic to be administered to the patient;
evaluating the rule set according to the patient data to determine if a user should be alerted;
alerting a user if the rule set indicates that a user should be alerted; and
sending the identity of the therapeutic to an associated system for documentation.

8. The method of claim 7, wherein the method further comprises administering the therapeutic to the patient, the patient data includes a first parameter that indicates if an action has been taken by a caregiver, and evaluating the rule set comprises determining that an alert is necessary if the first parameter indicates that the action has not been taken within a predetermined time period after administering the therapeutic.

9. The method of claim 7, evaluating the rule set according to the patient data to determine if the user should be alerted comprises determining, from at least the indicator, if the therapeutic should be administered to the patient.

10. The method of claim 9, wherein the indicator further represents a dosage of the therapeutic and determining if the therapeutic should be administered to the patient comprises determining that the therapeutic should not be administered to the patient if the dosage is not within a defined range.

11. The method of claim 10, wherein the patient data comprises a first parameter and determining if the therapeutic should be administered to the patient further comprises selecting the defined range according to the first parameter, such that a first defined range is used if the first parameter assumes a first value and a second defined range is used if the first parameter assumes a second value.

12. The method of claim 9, wherein determining if the therapeutic should be administered to the patient comprises determining that the therapeutic should not be administered to the patient if a dose of the therapeutic was prepared more than a threshold period of time before it is to be administered.

13. The method of claim 9, wherein the therapeutic is a first therapeutic, and determining if the therapeutic should be administered to the patient comprises determining that the first therapeutic should not be administered to the patient if a second therapeutic is currently being administered to the patient on an intravenous line through which the first therapeutic is to be administered.

14. The method of claim 9, wherein the patient data comprises a first parameter and determining if the therapeutic should be administered to the patient comprises determining that the therapeutic should not be administered to the patient if the first parameter assumes a first value.

15. The method of claim 14, wherein the first parameter is a categorical parameter representing a diagnosis of a medical condition for the patient.

16. The method of claim 14, wherein the first parameter is a categorical parameter representing a diagnosis of a medical condition for the patient within a predetermined time period before administration of the therapeutic.

17. The method of claim 14, wherein the first parameter is a categorical parameter representing administration of a dose of a medication within a predetermined time period before administration of the therapeutic.

18. The method of claim 7, further comprising altering at least one of the plurality of rule sets according to patient outcomes retrieved from the electronic health records database.

19. The method of claim 7, further comprising receiving an input from the user representing disagreement with the alert and altering at least one of the plurality of rule sets according to the input from the user.

20. A system comprising:
a processor;
a non-transitory computer readable medium, storing executable instructions, the executable instructions comprising:
an interface configured to receive a first set of patient data in real-time from at least one sensor monitoring vital signals of a patient during a surgical procedure and a second set of data in real-time from an anesthesia machine;
an anesthesia information management system (AIMS) interface configured to receive a third set of patient data from an AIMS during the surgical procedure;
a network interface configured to retrieve a fourth set of patient data associated with the patient from an electronic health records database;
an input interface that receives an indicator representing at least an identity of a therapeutic to be administered to the patient; and
a rule-based expert system, comprising a plurality of rule sets, each rule set determining if an alert should be provided to a user from at least one of the indicator, the first set of patient data, the second set of patient data, and the third set of patient data; and
a retraining engine that retrains at least a subset of the plurality of rule sets according to a set of labelled training samples, each training sample including at least a portion of the data comprising the indicator, the first set of patient data, the second set of patient data, and the third set of patient data, as well as a label derived from at least one of patient outcomes retrieved from the electronic health records database and disagreements indicated by users; and
an output device that provides the alert to the user if a rule set of the plurality of rule sets determines that the alert should be provided to the user, wherein the user can indicate disagreement with the alert via the input interface.
